(19) United States
(12) Patent Application Publication
(10) Pub. No.: US 2024/0238101 A1
(43) Pub. Date: Jul. 18, 2024

(54) DEVICE AND METHOD OF USING THE DEVICE FOR ASSESSING RANGE-OF-MOTION DURING HIP RESURFACING PROCEDURES

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Nicholas R. Turner, Devizes (GB); Matthew Cannell, Totley (GB); Michael D. Hollandsworth, Jr., Memphis, TN (US); Abel C. Kim, Cordova, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/522,493

(22) Filed: Nov. 29, 2023

Related U.S. Application Data
(60) Provisional application No. 63/479,776, filed on Jan. 13, 2023.

(51) Int. Cl. A61F 2/46

(52) U.S. Cl. CPC A61F 2/4657; A61F 2002/4632; A61F 2002/4668

(57) ABSTRACT

Disclosed herein is a device, and a method for using the device to measure the range-of-motion of a hip joint during a hip resurfacing surgery. The device comprises a temporary femoral component for rigidly affixing an array holder to the resurfaced femoral head to hold a primary array of tracking markers that are tracked through the range-of-motion by a computer-assisted surgical system (CASS).

### RELATED APPLICATIONS

This application claims the benefit of U.S. Provisional Patent Application No. 63/479,776, filed Jan. 13, 2023, entitled “Device and Method of Using the Device for Assessing Range-Of-Motion During Hip Resurfacing Procedures”, the contents of which are incorporated herein in their entirety.

### FIELD OF THE DISCLOSURE

The present disclosure relates generally to orthopedic procedures, for example, total joint replacement procedures, and, in particular, to total hip arthroplasty procedures.

### BACKGROUND

Orthopedic procedures to replace joints, for example, knees and hips, are well known and commonplace in today's society. Such procedures may require removal or reshaping of portions of a bone to accept an orthopedic implant to replace the original joint. For example, during a traditional total hip arthroplasty (THA), a surgeon needs to broach the femoral canal of the patient to accept an implant having a stem portion which is inserted into the femoral canal and a ball-portion of a ball and socket joint, which is joined with a socket portion that is implanted into the hip bone.

Total hip resurfacing arthroplasty is an alternative form of THA in which the majority of the femur is preserved. The head of the femur is resurfaced to accept an implant containing the ball portion of the hip joint and an acetabular implant is positioned in the hip bone containing the socket portion of the hip joint. One such system for hip resurfacing is the Birmingham Hip® Resurfacing System by Smith & Nephew®, Inc., which includes all system components necessary to perform the total hip resurfacing, namely, acetabular cups, femoral resurfacing implants and the surgical instrumentation required to complete the procedure.

Navigated total hip arthroplasty often includes predictive range-of-motion (ROM) capabilities for the entire implant construct that uses femoral version measurements based off broach and stem data. Traditional THA procedures may be performed with the assistance of a navigated or computer-assisted surgical platform which may perform the assessment of the range-of-motion of the joint prior to placement the implant components. However, during the resurfacing procedure, it is more difficult to assess the range-of-motion because of the lack of broach and stem data often used in traditional procedures. Therefore, it would be desirable to provide a way to easily assess the range-of-motion during the hip resurfacing procedure.

### SUMMARY OF THE DISCLOSURE

The device, and the method for using the device to measure the range-of-motion described herein comprise a temporary femoral component for rigidly affixing an array holder to the resurfaced femoral head to hold a primary array of tracking markers that are tracked through the range-of-motion by a computer-assisted surgical system (CASS).

The temporary femoral component comprises a generally hemispherically-shaped body having an interior surface sized and shaped to accept the resurfaced head of the femur. The temporary femoral component is affixed in place on the resurfaced femoral head and provides an attachment point for the primary array of tracking markers. In certain examples, a secondary array of tracking markers may be attached at other points on the femur.

Software running on the CASS is used to spatially track the arrays used to assess the range-of-motion. A display may be provided showing the surgeon the calculated range-of-motion. The CASS may also assist in the placement of the acetabular component. In the event that the range-of-motion needs to be adjusted, the acetabular component may be repositioned.

In a first example, a device for attaching a marker array to a femoral head comprises a generally hemispherically-shaped body having an interior surface contoured to match a surface of a femoral head prepared for a hip resurfacing procedure and a connection point defined in the body for accepting a holder for an array of tracking markers such that the array of tracking markers is rigidly held in place with respect to the femoral head.

In any preceding or subsequent example, the device further comprises a post extending into the interior of the hemispherically-shaped body, the post configured to be disposed within a hole in the femoral head defined along a longitudinal axis of the femoral head.

In any preceding or subsequent example, the device further comprises a groove defined on an outer surface of the hemispherically-shaped body to assist in the alignment of the body on the femoral head.

In any preceding or subsequent example, the device further comprises a hole, defined in the hemispherically-shaped body and a pin for placement within the hole, the pin extending into the femoral head to secure the hemispherically-shaped body to the femoral head.

In any preceding or subsequent example, the device further comprises wherein the holder for the tracking array is disposed in the connection point.

In any preceding or subsequent example, the device further comprises wherein the connection point comprises a recess configured to accept a proximal end of the holder for the tracking array.

In any preceding or subsequent example, the device further comprises the tracking array disposed at a distal end of the holder.

In an alternate example, a method of assessing range-of-motion of a hip joint comprises placing a generally hemispherically-shaped body on a femoral head resurfaced to accept a femoral component of a hip implant, connecting an array of tracking markers to the hemispherically-shaped body such that the array is rigidly secured in a predetermined position with respect to the femoral head and assessing the range-of-motion of the hip joint by spatially tracking the array of tracking markers.

In any preceding or subsequent example, the method further comprises determining that the range-of-motion of the hip joint is unacceptable, adjusting the position of an acetabular component of the hip implant and reassessing the range-of-motion of the hip joint.

In any preceding or subsequent example, the method further comprises wherein the range-of-motion is assessed using a computer-aided surgical system comprising a surgical computer, a tracking system coupled to the surgical computer, software, executed by the surgical computer for assessing the range-of-motion given an input of the spatial position of the tracking markers from the tracking system and a display for displaying results of the assessment of the range-of-motion.

In any preceding or subsequent example, the method further comprises wherein an initial position of the hip joint is registered with the computer-aided surgical system using a secondary array of tracking markers rigidly attached to a proximal femur of the hip joint.

In any preceding or subsequent example, the method further comprises wherein the software compares the spatial position of the tracking markers in the primary array to the initial position of the hip joint to determine the range-of-motion.

In any preceding or subsequent example, the method further comprises wherein the computer-aided surgical system visualizes the range-of-motion on the display.

In any preceding or subsequent example, the method further comprises wherein the visualization of the range-of-motion comprises a dynamic display animating movement of the femur as the hip joint is reduced.

In any preceding or subsequent example, the method further comprises wherein the software performs the function of recommending a position of the acetabular component of the hip implant to improve the range-of-motion.

In any preceding or subsequent example, the method further comprises wherein the hemispherically-shaped body has an interior surface contoured to match a surface of the resurfaced femoral head.

In any preceding or subsequent example, the method further comprises wherein the hemispherically-shaped body has a post extending from an interior surface of the hemispherically-shaped body, the post configured to be disposed within a hole in the femoral head defined along a longitudinal axis of the femoral head.

In any preceding or subsequent example, the method further comprises wherein the hemispherically-shaped body has a groove defined on an outer surface thereof to assist in the alignment of body on the femoral head.

In any preceding or subsequent example, the method further comprises wherein the hemispherically-shaped body defines a hole therein configured to accept a pin for placement within the hole, the pin extending into the femoral head to secure the hemispherically-shaped body to the femoral head.

In an alternate example, a computer-aided surgical system comprises a surgical computer, a tracking system coupled to the surgical computer, software, executed by the surgical computer for assessing the range-of-motion given an input of the spatial position of an array of tracking markers and a display for displaying results of the assessment of the range-of-motion, wherein the tracking system tracks the array of tracking markers rigidly attached to a femoral head resurfaced to accept a femoral component of a hip implant, the array being rigidly connected to the femoral head using a hemispherically-shaped body having an inner surface contoured to match a contour of the resurfaced femoral head, the hemispherically-shaped body rigidly attached to the femoral head.

Examples of the present disclosure provide numerous advantages. For example, the procedure provides a method for assessing the range-of-motion that is significantly simpler than prior-art methods and which may be easily accomplished within the framework of the workflow of a typical procedure, without major interruption to the workflow. The device and method are designed to work with a CASS system and existing registration procedures. The CASS may provide a visualization of the range-of-motion and may also assist in the placement of the acetabular component to achieve an optimal range-of-motion.

Further features and advantages of at least some of the examples of the present disclosure, as well as the structure and operation of various examples, are described in detail below with reference to the accompanying drawings.

### BRIEF DESCRIPTION OF THE DRAWINGS

By way of example, specific examples of the disclosed system and method will now be described, with reference to the accompanying drawings, in which:

FIG. 1 shows an exemplary implant use during hip resurfacing surgery comprising a femoral component and an acetabular component.

FIG. 2 is a depiction of a femoral head being prepared for resurfacing and for acceptance of the femoral component.

FIG. 3 is a depiction of the femur after femoral head preparation.

FIG. 4 is a depiction of the femoral head having the permanent femoral component disposed thereon.

FIG. 5 is a depiction of the preparation of the acetabulum for acceptance of the acetabular component.

FIGS. 6(a-b) show a perspective view and a top view respectively of the temporary femoral component of the present examples.

FIGS. 7(a-b) show the temporary femoral component in place on a prepared femoral head and a cross-section of the temporary femoral component in place on the prepared femoral head, respectively.

FIGS. 8(a-b) show the array holder in place in the temporary femoral component and a cross-sectional view of the array holder in place in the temporary femoral component, respectively.

FIG. 9 shows another view of the temporary femoral component in place on the femoral head, the array holder connected to the temporary femoral component and a connection mechanism connected to the distal end of the holder.

FIG. 10 shows the secondary array in situ on the proximal femur.

FIG. 11 is a block diagram depicting an exemplary computer-aided surgical system.

FIG. 12 is a flowchart showing a modified surgical workflow including steps necessary to assess the range-of-motion of the hip joint utilizing the device of the present examples.

### DEFINITIONS

For the purposes of this disclosure, the term “implant” is used to refer to a permanent or temporary prosthetic device or structure manufactured to replace or enhance a biological structure. For example, in a total hip replacement procedure a prosthetic acetabular cup (implant) is used to replace or enhance a patients' worn or damaged acetabulum. While the term “implant” is generally considered to denote a man-made structure (as contrasted with a transplant), for the purposes of this specification an implant can include a biological tissue or material transplanted to replace or enhance a biological structure.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly stated. For example, a reference to a surgeon could also apply, in some examples to a technician or nurse.

### DETAILED DESCRIPTION

The device and method disclosed herein provides intra-operative information to a surgeon to assist the surgeon in determining the range-of-motion of a hip joint undergoing a hip resurfacing procedure.

This disclosure is not limited to the particular systems, devices and methods described, as these may vary. The terminology used in the description is for the purpose of describing the particular versions or examples only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

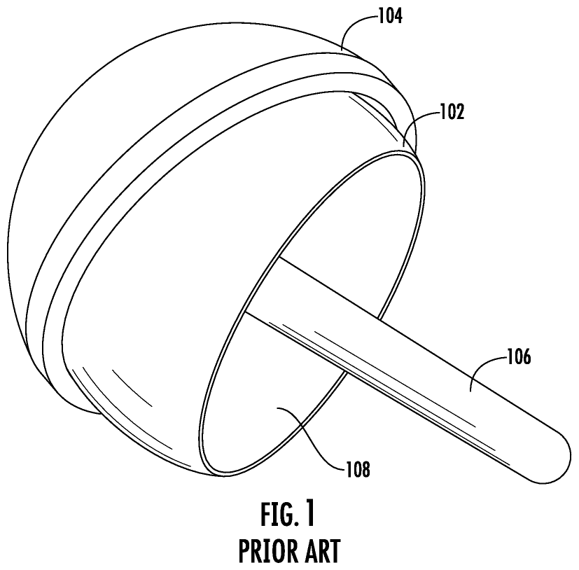

The device and method disclosed herein is best explained in terms of its use during a typical hip resurfacing procedure. FIG. 1 shows a typical implant use during the hip resurfacing procedure. During the procedure, the head of the femur is shaped to accept femoral component 102. A hole is drilled through the longitudinal axis of the head of the femur to accept post 106 and the head of the femur is shaped using one or more bone reamers to conform to the interior surface 108 of the femoral component 102. The acetabulum is reamed to remove any remaining cartilage and the acetabular component 104 is fitted into the pelvic bone using a friction fit.

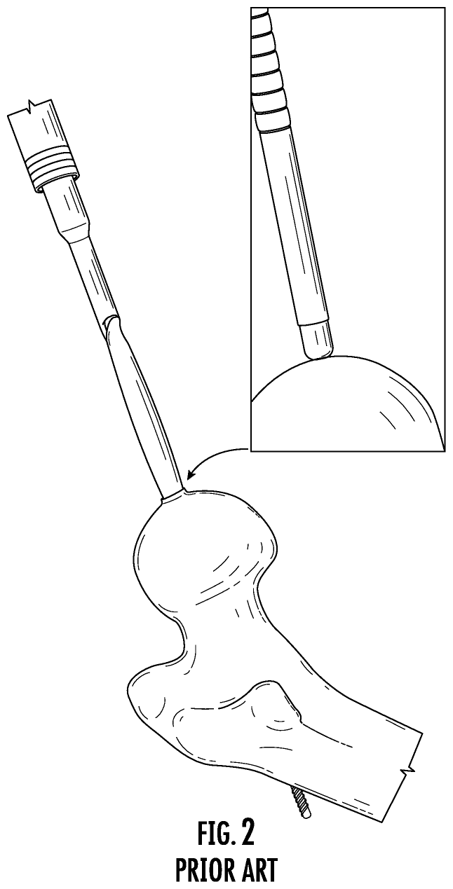

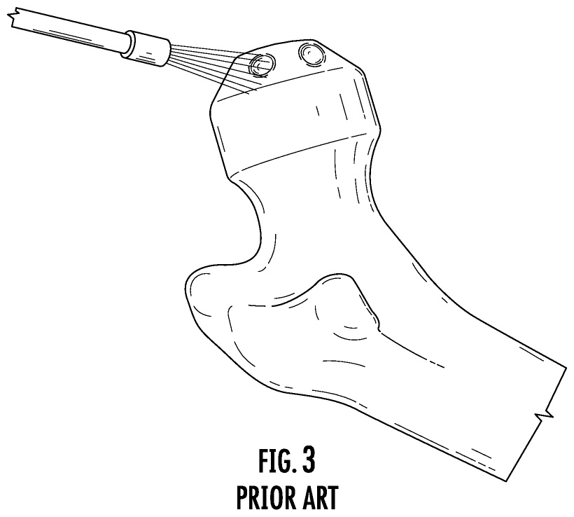

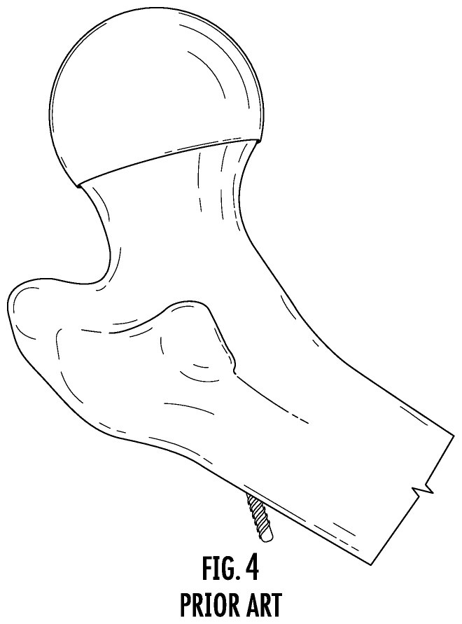

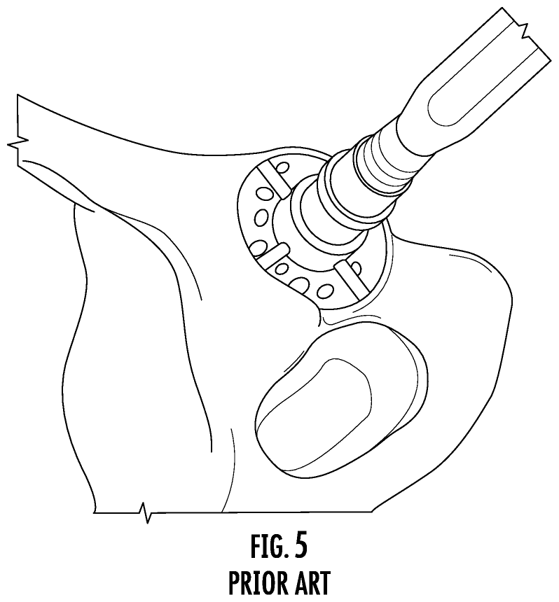

FIG. 2 shows the drilling of the hole along the longitudinal axis of the femoral head. Once the hole is drilled, a post is placed in the hole which will guide the bone reamers during the process of shaping the femoral head. FIG. 3 shows the shaped femoral head, wherein the shape of the femoral head conforms to the interior surface 108 of the femoral component 102. FIG. 4 shows the femoral component 102 in place on the femoral head. The femoral component 102 may be secured on the femoral head using a cement. FIG. 5 shows the reaming of the acetabulum to remove any remaining cartilage such that the acetabular component 104 can be placed.

Once the femoral head has been prepared, as shown in FIG. 3, and prior to the placement of the femoral component, as shown in FIG. 4 the device of the present disclosure is placed on the femoral head as will be described to assess the potential range-of-motion, given the placement of the acetabular component 104.

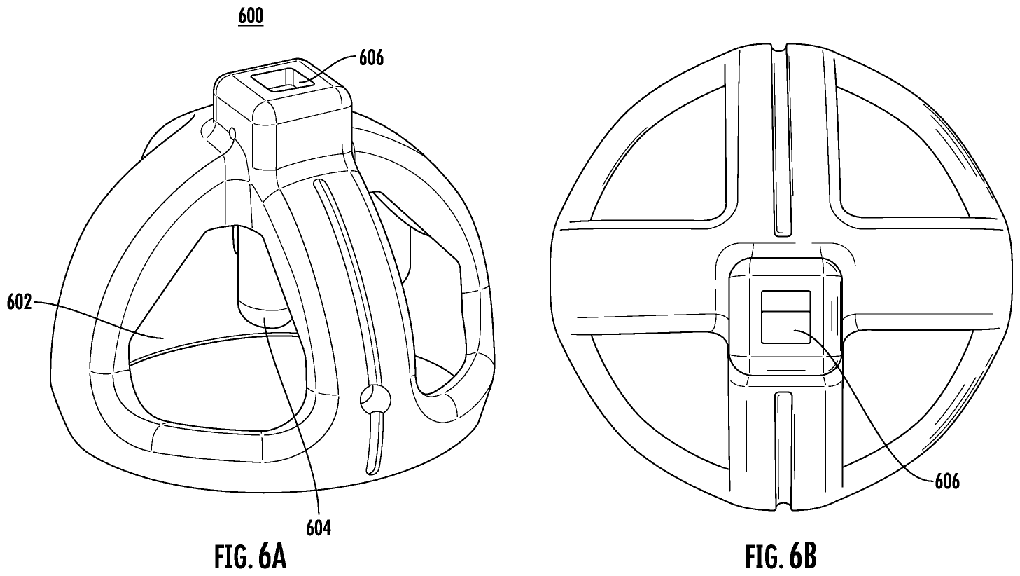

The device of the present disclosure comprises a temporary femoral component 600 as shown in perspective view in FIG. 6(a) and in a top view in FIG. 6(b). Once the femoral head has been prepared as previously described, the temporary femoral component 600 is generally cap-shaped or hemispherically-shaped and is positioned onto the prepared femoral head. The temporary femoral component 600 has an internal surface 602 that conforms to the contour of the prepared femoral head and is substantially identical to the interior surface 108 of femoral component 102. Temporary femoral component 600 is also provided with post 604 that extends from an interior surface of temporary femoral component 600 and is disposed into the hole drilled in the femoral head once the temporary femoral component 600 is in place. In various examples, temporary femoral component 600 may be composed of any material, for example, metal or plastic.

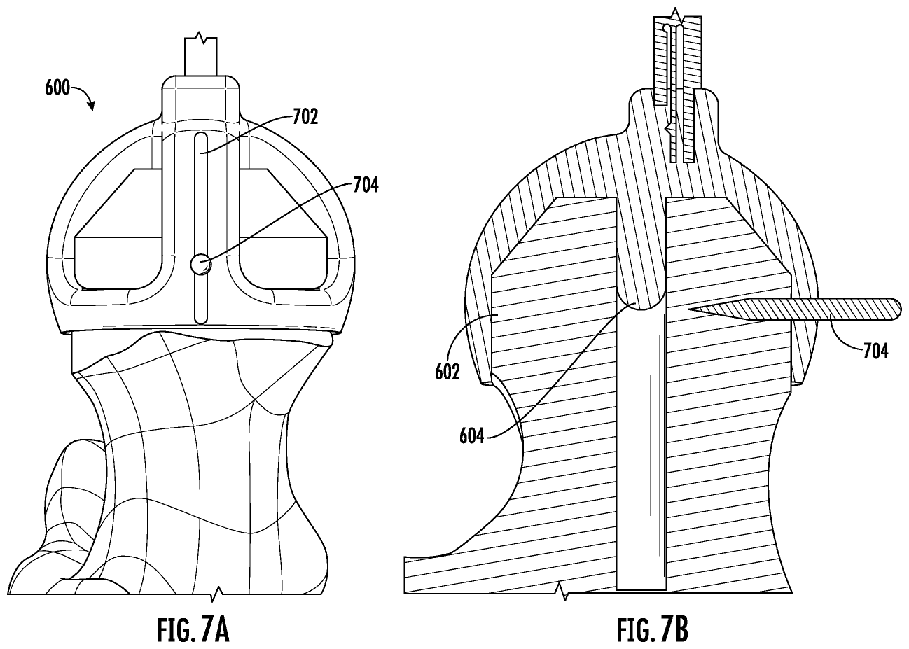

FIG. 7(a) shows the temporary femoral component 600 in place on the head of the femur. Temporary femoral component 600 may be provided with a groove 702 which is defined in the surface of temporary femoral component 600 to assist in the alignment of temporary femoral component 600 on the head of the femur. Temporary femoral component 600 may be rotated on the femoral head until groove 702 is aligned such as to be central to the neck of the native femur. FIG. 7(b) shows a cutaway view of temporary femoral component 600 in place on the head of the femur. In some examples, temporary femoral component 600 may be held in place by pin 704 which is driven through a hole defined in temporary femoral component 600 and into the femoral head to secure temporary femoral component 600 to the femoral head.

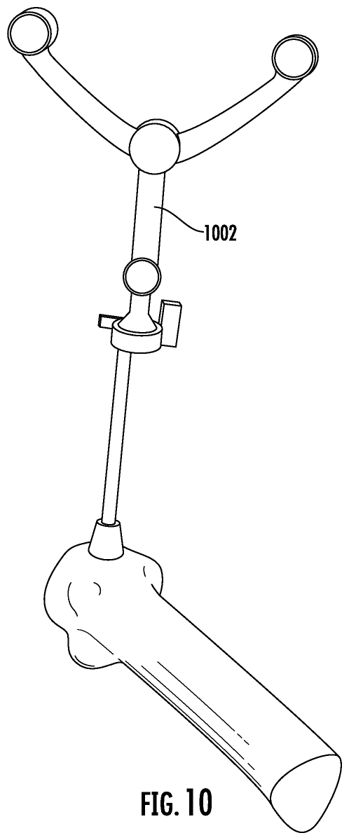

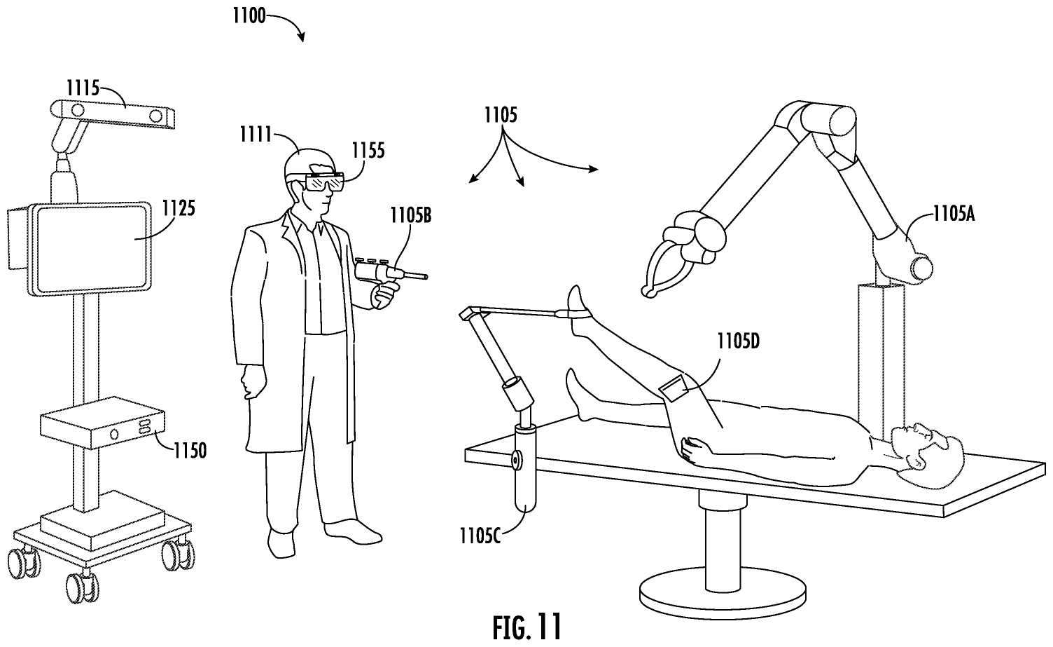

Temporary femoral component 600 is configured with a recess 606, as shown in FIGS. 6(a) and 6(b), to accept a holder 800 for a primary array of markers which are tracked by a camera of a computer-assisted surgical system (CASS) 1100, shown in FIG. 11. The purpose of the array placement on the holder is to determine the position of the milled femoral head relative to the eventual final implant, and therefore to allow a virtual range-of-motion to be calculated. This can be accomplished by determining the relative positions of the primary array and a secondary array 1002, as shown in FIG. 10. Note that it is not necessary to have the primary array in place when the range-of-motion is assessed. Holder 800 and the primary array could be placed, position recorded, and removed, and the range-of-motion could be assessed at any time.

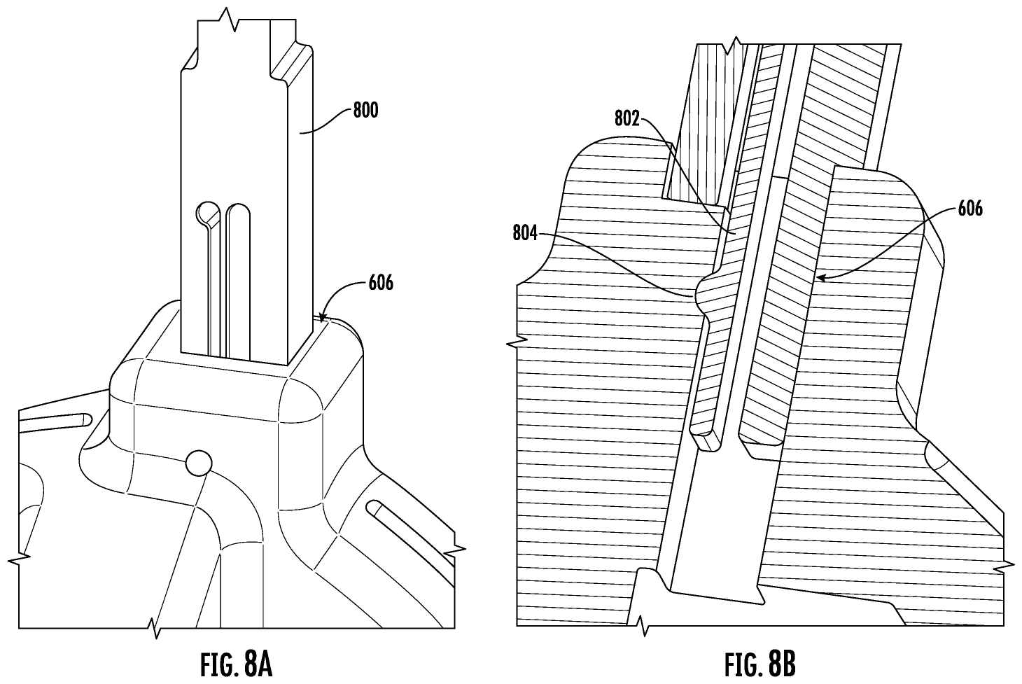

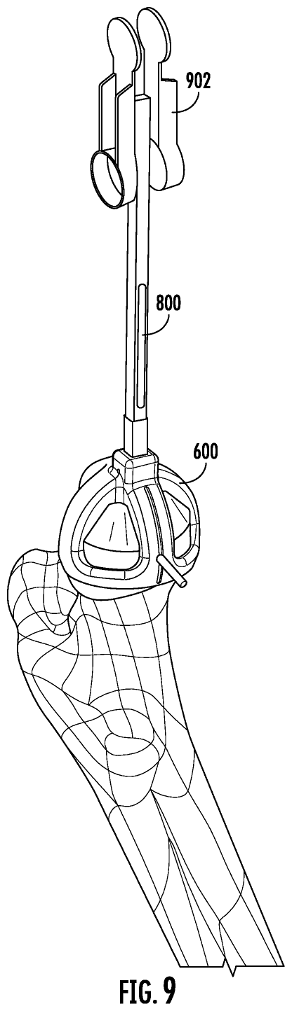

Details of one example of the recess 606 are shown in perspective view in FIG. 8(a) and in cutaway view in FIG. 8(b), showing holder 800 in situ. In the example shown, holder 800 is configured with a spring mechanism 802 having an enlarged portion which, when holder 800 is engaged with temporary femoral component 600, is confined within recess 804. This configuration is designed to rigidly secure holder 800 in a specific orientation with respect to the femoral head. As would be realized by one of skill in the art, the actual configuration of the interface between holder 800 and temporary femoral component 600 may be of any design capable of securely holding an array of markers in a predefined position with respect to the prepared femoral head. FIG. 9 shows holder 800 properly engaged with temporary femoral component 600. Holder 800 may be configured with connection mechanism 902 to which a primary array of markers may be connected. The positioning of temporary femoral component 600 in the proper position with respect to the femoral head and the secure fastening of holder 800 to temporary femoral component 600 such that it is in a predetermined position with respect to the femoral head is crucial to the proper assessment of the range-of-motion.

To properly assess the range-of-motion, it may first be necessary to register the femur with the CASS system 1100 such as to establish a baseline positioning of the femur. To accomplish this, the femur may be configured with a secondary array 1002 of tracking markers which may be connected to the proximal femur, as shown in FIG. 10. The secondary array 1002 may be fitted to the proximal femur by securing a magnetic array holder fixed to the proximal femur by small spikes and a screw. The secondary array 1002 is used to register the native femoral anatomy prior to preparation of the femoral head. Anatomical references provided by the primary array and secondary array 1002 are stored by the CASS 1100 and the secondary array 1002 is then removed to allow for the normal surgical workflow described with respect to FIGS. 1-5. After shaping the femoral head and attaching the temporary femoral component 600, the secondary array 1002 may be reattached to measure femoral orientation and version. By utilizing the positional data from both the secondary array 1002 and the primary array held by holder 800, the CASS 1100 can determine the optimal orientation for the acetabular component 104. In the event that the range-of-motion is less than optimal, it may be necessary to reposition acetabular component 104.

The systems and methods described herein may be implemented as software application running on a computing system interfaced with a navigated or robotic-assisted surgical platform or, alternatively, as part of the software of the navigated or robotic-assisted surgical platform.

FIG. 11 provides an illustration of an exemplary computer-assisted surgical system (CASS) 1100, with which the system and method of the disclosure may be implemented. CASS system 1100 uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. CASS system 1100 often employs various forms of computing technology to perform a wide variety of standard and minimally invasive surgical procedures and techniques. Moreover, these systems allow surgeons to more accurately plan, track and navigate the placement of instruments and implants relative to the body of a patient, as well as to conduct pre-operative and intra-operative body imaging.

An effector platform 1105 positions surgical tools relative to a patient during surgery. The exact components of the effector platform 1305 will vary, depending on the example employed. For example, for a knee surgery, the effector platform 1105 may include an end effector 1105B that holds surgical tools or instruments during their use. The end effector 1105B may be a handheld device or instrument used by the surgeon (e.g., a NAVIO® or CORI® hand piece or a cutting guide or jig) or, alternatively, the end effector 1105B can include a device or instrument held or positioned by a robotic arm 1105A.

The effector platform 1105 can include a limb positioner 1105C for positioning the patient's limbs during surgery. One example of a limb positioner 1105C is the SMITH & NEPHEW SPIDER2 system. The limb positioner 1105C may be operated manually by the surgeon or alternatively change limb positions based on instructions received from the surgical computer 1150 (described below).

Resection equipment (not shown in FIG. 11) performs bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, and laser ablation systems. In some examples, the resection equipment is held and operated by the surgeon during surgery. In other examples, the effector platform 1105 may be used to hold the resection equipment during use.

The effector platform 1105 can also include a cutting guide or jig 1105D that is used to guide saws or drills used to resect tissue during surgery. Such cutting guides or jigs 1105D can be formed integrally as part of the effector platform 1105 or robotic arm 1105A or cutting guides can be separate structures that can be removably attached to the effector platform 1105 or robotic arm 1105A. The effector platform 1105 or robotic arm 1105A can be controlled by the CASS system 1100 to position a cutting guide or jig 1105D adjacent to the patient's anatomy in accordance with a pre-operatively or intra-operatively developed surgical plan such that the cutting guide or jig will produce a precise bone cut in accordance with the surgical plan.

The tracking system 1115 uses one or more sensors to collect real-time position data that locates the patient's anatomy and surgical instruments. For example, for TKA procedures, the tracking system 1115 may provide a location and orientation of the end effector 1105B or various arrays of tracking markers during the procedure. In addition to positional data, data from the tracking system 1115 can also be used to infer velocity/acceleration of anatomy/instrumentation, which can be used for tool control. In some examples, the tracking system 1115 may use a tracker array attached to the end effector 1105B to determine the location and orientation of the end effector 1105B. The position of the end effector 1105B may be inferred based on the position and orientation of the tracking system 1115 and a known relationship in three-dimensional space between the tracking system 1115 and the end effector 1105B. Various types of tracking systems may be used in various examples of the present disclosure including, without limitation, Infrared (IR) tracking systems, electromagnetic (EM) tracking systems, video or image-based tracking systems, and ultrasound registration and tracking systems.

The display 1125 provides graphical user interfaces (GUIs) that display images the user interface of the navigated surgical platform, as well other information relevant to the surgery. For example, the display 1125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 1125 may include, for example, one or more computer monitors. As an alternative or supplement to the display 1125, one or more members of the surgical staff may wear an augmented reality (AR) Head Mounted Device (HMD). For example, in FIG. 11 the surgeon 1111 is wearing an AR HMD 1155 that may, for example, overlay pre-operative image data on the patient or provide surgical planning suggestions. Various example uses of the AR HMD 1155 in surgical procedures are detailed in the sections that follow.

Surgical computer 1150 provides control instructions to various components of CASS system 1100, collects data from those components, and provides general processing for various data needed during surgery. In some examples, the surgical computer 1150 is a general-purpose computer. In other examples, the surgical computer 1150 may be a parallel computing platform that uses multiple central processing units (CPUs) or graphics processing units (GPU) to perform processing. In some examples, the surgical computer 1150 is connected to a remote server over one or more computer networks (e.g., the Internet). The remote server can be used, for example, for storage of data or execution of computationally intensive processing tasks.

In various examples, the systems and methods disclosed herein may be implemented as a software application executing on surgical computer 1150 integrated with or separate from the software controlling the navigated surgical platform. Also, in various examples, the systems and methods disclosed herein may be implemented as a software application executing on a computing platform other than surgical computer 1150, such as a laptop or tablet computing device. The computing device executing systems and methods disclosed herein may be in communication with surgical computer 1150.

With respect to the present disclosure, tracking system 1115 may be used to track the position of the primary and secondary arrays of markers. Software executing on surgical computer 1150 may register the initial position of the femur using the secondary array. Once the femoral head has been prepared and the temporary femoral component 600 properly affixed thereon, the range-of-motion may be assessed using the primary array attached to the temporary femoral component 600 and/or the secondary array. In some examples, the range of motion may be visualized on display 1125. The visualization may, in some examples, comprise a static display of the assessment of the range-of-motion, or, in other examples, may comprise a dynamic display showing movement of the femur as the joint is reduced. In some examples, the software executing on surgical computer 1150 may provide suggestions or recommendations for repositioning of the acetabular component 104 to obtain an optimal range-of-motion.

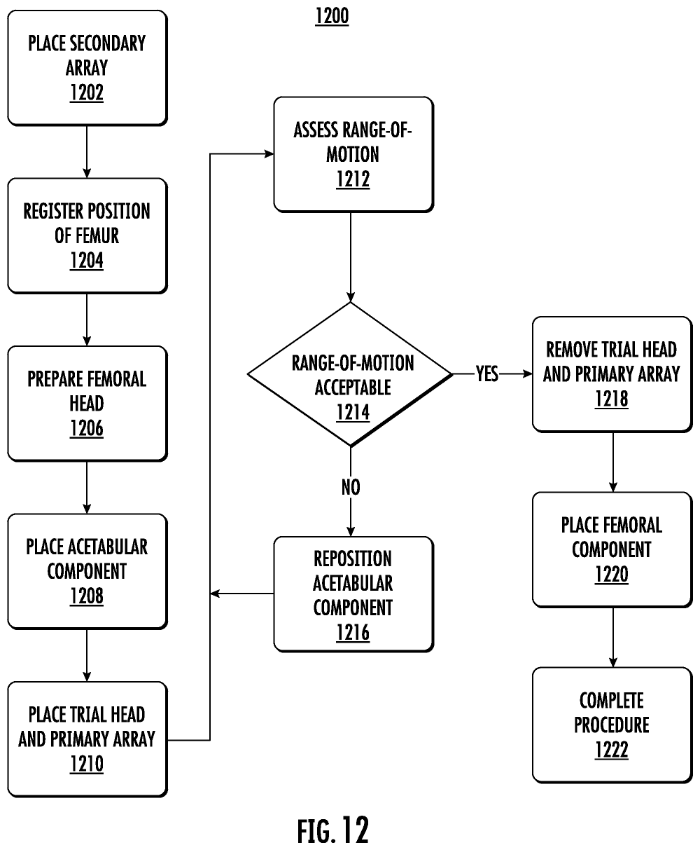

FIG. 12 is a flowchart showing the workflow the surgical procedure including the steps introduced by the examples disclosed herein. At step 1202, a secondary array 1002 is placed on the proximal femur, and at step 1204, the secondary array is used to register the native femoral anatomy with the CASS system 1100. At step 1206, the femoral head is prepared for acceptance of the femoral component 102 in accordance with the normal workflow of the hip resurfacing procedure. At step 1208, the acetabular component 104 is placed. At step 1210, the temporary femoral component 600 is placed on the prepared femoral head and the primary array is attached to the temporary femoral component 600. In some examples, it may be possible to place the holder 800 for the marker array directly in the hole drilled along the longitudinal axis of the femoral head, thus eliminating the need for the placement of the temporary femoral component 600. It should be noted that steps 1206, 1208 and 1210 may be performed in any order based on the preferences of the surgeon.

At step 1212, the range of motion is assessed with the assistance of CASS 1100. As previously mentioned, under control of software running on surgical computer 1150, the tracking system 1115 spatially tracks the position of the primary array and/or the secondary array to provide an assessment of the range-of-motion, which may be visualized on surgical display 1150. At decision point 1214, it is determined if the range-of-motion is acceptable. If the range-of-motion needs to be adjusted, at step 1216, the acetabular component may be repositioned. CASS 1100 may provide guidance as to adjustments of the acetabular component that will provide an improved range-of-motion. Controls and return to step 1212 for the range-of-motion is again assessed with the assistance of CASS 1100. Steps 1212, 1214 and 1216 may be iterated any number of times until an acceptable range-of-motion is determined at step 1214. Once an acceptable range-of-motion is achieved at step 1214, control proceeds step 1218, wherein the temporary femoral component 600 and primary array are removed from the femoral head. At step 1220, the permanent femoral component is placed on the femoral head and, at step 1222 the procedure is complete.

Various examples of a system and method have been described herein to provide a surgeon performing hip resurfacing procedure with a device and method for assessing the range-of-motion of the joint, given the placement of the acetabular component 104.

As would be realized by one of skill in the art, the disclosed examples have been explained in terms of a surgical procedure using the Birmingham Hip® Resurfacing System by Smith & Nephew®, Inc. However, the examples are equally applicable to surgeries performed utilizing other systems from other manufacturers.

Therefore, the present disclosure is not meant to be limited in terms of the particular examples described in this application, which are intended as illustrations of various features. Many modifications and variations can be made without departing from the spirit and scope of the disclosure, as will be apparent to those skilled in the art. Functionally equivalent devices and methods contemplated to be within the scope of the disclosure, in addition to those enumerated herein, will be apparent to those skilled in the art from the foregoing descriptions. It is to be understood that this disclosure is not limited to particular devices, methods, or systems. It is also to be understood that the terminology used herein is for the purpose of describing particular examples only and is not intended to be limiting.

1. A device for attaching a marker array to a femoral head comprising:
a generally hemispherically-shaped body having an interior surface contoured to match a surface of a femoral head prepared for a hip resurfacing procedure; and
a connection point defined in the body for accepting a holder for an array of tracking markers such that the array of tracking markers is rigidly held in place with respect to the femoral head.

2. The device of claim 1, further comprising:
a post extending from the hemispherically-shaped body configured to be disposed within a hole in the femoral head defined along a longitudinal axis of the femoral head.

3. The device of claim 2, further comprising:
a groove defined on an outer surface of the hemispherically-shaped body to assist in the alignment of body on the femoral head.

4. The device of claim 3, further comprising:
a hole, defined in the hemispherically-shaped body; and
a pin for placement within the whole, the pin extending into the femoral head to secure the hemispherically-shaped body to the femoral head.

5. The device of claim 1, further comprising:
the holder for the tracking array disposed in the connection point.

6. The device of claim 5, wherein the connection point comprises:
a recess configured to accept a proximal end of the holder for the tracking array.

7. The device of claim 6, further comprising:
the tracking array disposed at a distal end of the holder.

8. A method of assessing range-of-motion of a hip joint comprising:
placing a hemispherically-shaped body on a femoral head resurfaced to accept a femoral component of a hip implant;
connecting an array of tracking markers to the hemispherically-shaped body such that the array is rigidly secured in a predetermined position with respect to the femoral head; and
assessing the range-of-motion of the hip joint by spatially tracking the array of tracking markers.

9. The method of claim 8, further comprising:
determining that the range-of-motion of the hip joint is unacceptable;
adjusting positioning of an acetabular component of the hip implant; and
reassessing the range-of-motion of the hip joint.

10. The method of claim 9, wherein the range-of-motion is assessed using a computer-aided surgical system comprising:
a surgical computer;
a tracking system coupled to the surgical computer;
software, executed by the surgical computer for assessing the range-of-motion given an input of the spatial position of the tracking markers; and
a display for displaying results of the assessment of the range-of-motion.

11. The method of claim 10, wherein an initial position of the hip joint is registered with the computer-aided surgical system using a secondary array of tracking markers rigidly attached to a proximal femur of the hip joint.

12. The method of claim 10, wherein the software compares the spatial position of the tracking markers to the initial position of the hip joint to determine the range-of-motion.

13. The method of claim 10, wherein the computer-aided surgical system visualizes the range-of-motion on the display.

14. The method of claim 13, wherein the visualization of the range-of-motion comprises a dynamic display animating movement of the femur as the hip joint is reduced.

15. The method of claim 10, wherein the software performs the function of:
recommending a position of the acetabular component of the hip implant to improve the range-of-motion.

16. The method of claim 8, wherein the hemispherically-shaped body has an interior surface contoured to match a surface of the resurfaced femoral head.

17. The method of claim 8, wherein the hemispherically-shaped body has a post extending from the hemispherically-shaped body configured to be disposed within a hole in the femoral head defined along a longitudinal axis of the femoral head.

18. The method of claim 8, wherein the hemispherically-shaped body has a groove defined on an outer surface thereof to assist in the alignment of body on the femoral head.

19. The method of claim 8, wherein the hemispherically-shaped body further comprising defines a hole therein configured to accept a pin for placement within the hole, the pin extending into the femoral head to secure the hemispherically-shaped body to the femoral head.

20. A computer-aided surgical system comprising:
a surgical computer;
a tracking system coupled to the surgical computer;
software, executed by the surgical computer for assessing the range-of-motion given an input of the spatial position of the tracking markers; and
a display for displaying results of the assessment of the range-of-motion;
wherein the tracking system tracks an array of tracking markers rigidly attached to a femoral head resurfaced to accept a femoral component of a hip implant, the array being rigidly connected to the femoral head using a hemispherically-shaped body having an inner surface contoured to match a contour of the resurfaced femoral head, the hemispherically-shaped body rigidly attached to the femoral head.
